(19) United States
(12) Patent Application Publication
(10) Pub. No.: US 2002/0064803 A1
(43) Pub. Date: May 30, 2002

(54) Novel antibody compositions for preparing enriched eosinophil preparations

(76) Inventors: Terry E. Thomas, Vancouver (CA); Carolyn Horrocks, Stockholm (SE)

Correspondence Address: KILPATRICK STOCKTON LLP, Attn: John S. Pratt, Esq., Suite 2800, 1100 Peachtree Street, Atlanta, GA, 30309-4530, US

(21) Appl. No.: 09/853,892

(22) Filed: May 11, 2001

Related U.S. Application Data
(60) Provisional application No. 60/203,484, filed on May 11, 2000.

(51) Int. Cl. G01N 33/567; C07K 16/46

(52) U.S. Cl. 435/7.21; 530/387.3

(57) ABSTRACT

The present invention relates to antibody composition that are useful in preparing enriched eosinophil cell preparations. The invention also relates to kits for carrying out the processes and to the cell preparations prepared by the processes.

This application claims the benefit under 35 USC §119(e) from U.S. Provisional patent application Ser. No. 60/203,484, filed May 11, 2000.

### FIELD OF THE INVENTION

The present invention relates to novel antibody compositions, and processes and kits for preparing cell preparations enriched for eosinophils.

### BACKGROUND OF THE INVENTION

Eosinophilic granulocytes constitute 2-5% of peripheral blood leukocytes. Together with neutrophils, eosinophils form the polymorphonuclear (PMN) fraction of peripheral blood leukocytes which pellets with the erythrocytes during a standard ficoll density separation. They are distinguished phenotypically from neutrophils by their expression of CD9 and absence of CD16. Eosinophils are only weakly phagocytic and are less efficient than neutrophils at intracellular killing. Their main function is the release of toxic granules for the extracellular killing of parasites. They are also involved in the pathology of atopic diseases such as eczema and asthma.

The initial step of procedures to isolate eosinophils involves a standard ficoll separation to eliminate the majority of mononuclear cells which remain at the ficoll/plasma interface. The cells in the pellet include erythrocytes, granulocytes and a minor portion of the mononuclear cells. The pellet is resuspended and the erythrocytes are eliminated in a lysis step. The cell suspension after lysis is typically 90% neutrophils, 5% eosinophils and 5% mononuclear cells. Current procedures for the further enrichment of eosinophils involve removal of neutrophils by target with anti-CD16 antibody followed by selective depletion using immunoadsorption techniques (panning, affinity columns or magnetic bead separations) or Fluorescent Activated Cell Sorting (FACS). Such technique do not remove the mononuclear cells (approximately 5% of the cells) which become a significant proportion of the cells once the neutrophils are removed.

There is a need in the art to develop new methods for the enrichment of eosinophils.

### SUMMARY OF THE INVENTION

The inventors have developed antibody compositions for use in preparing cell preparations highly enriched for eosinophils from the mixture of cells which pellet in a standard ficoll density separation of whole peripheral blood. Antibodies to mononuclear cells and residual erythroid cells (did not lyse) drastically increase the purity of eosinophils in the final enriched fraction.

To enrich for eosinophils, the antibodies in the antibody composition are specific for selected markers associated with cells that are not eosinophils thereby allowing them to be removed from the cell preparation. In particular, the present inventors have found using a negative selection technique that an antibody composition containing antibodies specific for (a) CD16; (b) CD2 and/or CD3 and/or both CD4 and CD8; (c) CD19 and/or CD20 and/or CD21 and/or CD22 and/or CD24 and/or IgG; and (d) CD14 and/or CD36 gives a cell preparation highly enriched for eosinophils. Optionally, the antibody composition additionally includes antibodies to CD33, CD56, IgM, CD41 or glycophorin A.

The enrichment and recovery of eosinophils using the antibody compositions of the invention in a negative selection technique has many advantages over conventional positive selection techniques. Highly enriched cell preparations can be obtained using a single step. The cells obtained using the antibody composition of the invention are not labeled or coated with antibodies or modified making them highly suitable for many uses.

The present invention also relates to a negative selection process for enriching and recovering eosinophils in a sample comprising (1)reacting the sample with an antibody composition containing antibodies capable of binding to the antigens (a) CD16; (b) CD2 and/or CD3 and/or both CD4 and CD8; (c) CD19 and/or CD20 and/or CD21 and/or CD22 and/or CD24 and/or IgG; and (d) CD14 and/or CD36 under conditions permitting the formation of conjugates between the antibodies and cells in the sample having the antigens (a) CD16; (b) CD2 and/or CD3 and/or both CD4 and CD8; (c) CD19 and/or CD20 and/or CD21 and/or CD22 and/or CD24 and/or Ig; and (d) CD14 and/or CD36 on their surfaces; (2) removing the conjugates; and (3) recovering a cell preparation which is enriched in eosinophils.

The present invention also relates to a kit useful in preparing a cell preparation enriched in eosinophils comprising antibodies specific for the antigens (a) CD16; (b) CD2 and/or CD3 and/or both CD4 and CD8; (c)CD19 and/or CD20 and/or CD21 and/or CD22 and/or CD24 and/or Ig; and (d) CD14 and/or CD36, and instructions for preparing a cell preparation enriched in eosinophils.

The invention further relates to cell preparations obtained in accordance with the processes of the invention. The invention still further contemplates a method of using the antibody compositions of the invention in negative selection methods to recover a cell preparation which is enriched in eosinophils.

These and other aspects of the present invention will become evident upon reference to the following detailed description and attached drawings. In addition, reference is made herein to various publications, which are hereby incorporated by reference in their entirety.

### BRIEF DESCRIPTION OF THE DRAWINGS

The invention will now be described with reference to the accompanying drawings, in which:

FIG. 1 is a schematic representation of magnetic cell labeling using tetrameric antibody complexes and colloidal dextran iron.

### DETAILED DESCRIPTION OF THE INVENTION

I. Antibody Compositions

As hereinbefore mentioned, the invention relates to an antibody compositions for preparing cell preparations enriched in eosinophils. In one aspect, the antibody composition is for enriching eosinophils and comprises antibodies specific for the antigens (a) CD16; (b) CD2 and/or CD3 and/or both CD4 and CD8; (c) CD19 and/or CD20 and/or CD21 and/or CD22 and/or CD24 and/or Ig; and (d) CD14 and/or CD36, which are present on the surface of non-eosinophils.

The above antibody compositions may additionally include other antibodies such antibodies that can bind to the antigens CD33, CD56, IgM, CD41 or glycophorin A. In a specific embodiment, the antibody composition comprises antibodies specific for the antigens CD16, CD2, CD19 and CD14 and optionally CD56 and glycophorin A. Antibodies useful in the invention may be prepared as described below using techniques known in the art or may be obtained from commercial sources.

One skilled in the art will appreciate that in addition to the antibodies listed above, the eosinophil enrichment cocktail may additionally include other antibodies that are specific for antigens on the surface of non-eosinophils. The selection of the antibodies can depend on many factors including the nature of the sample to be enriched.

Within the context of the present invention, antibodies are understood to include monoclonal antibodies and polyclonal antibodies, antibody fragments (e.g., Fab, and F(ab′)₂) and chimeric antibodies. Antibodies are understood to be reactive against a selected antigen on the surface of a cell if they bind with an appropriate affinity (association constant), e.g. greater than or equal to 10⁷ M⁻¹.

Polyclonal antibodies against selected antigens on the surface of cells may be readily generated by one of ordinary skill in the art from a variety of warm-blooded animals such as horses, cows, various fowl, rabbits, mice, hamsters, or rats. For example, a mammal, (e.g., a mouse, hamster, or rabbit) can be immunized with an immunogenic form of an antigen which elicits an antibody response in the mammal. Techniques for conferring immunogenicity on an antigen include conjugation to carriers or other techniques well known in the art. For example, the antigen can be administered in the presence of adjuvant. The progress of immunization can be monitored by detection of antibody titers in plasma or serum. Following immunization, antisera can be obtained and polyclonal antibodies isolated from the sera.

Monoclonal antibodies are preferably used in the antibody compositions of the invention. Monoclonal antibodies specific for selected antigens on the surface of non-eosinophils may be readily generated using conventional techniques. For example, monoclonal antibodies may be produced by the hybridoma technique originally developed by Kohler and Milstein 1975 (Nature 256, 495-497; see also U.S. Pat. Nos. RE 32,011, 4,902,614, 4,543,439, and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988). Other techniques may also be utilized to construct monoclonal antibodies (for example, see William D. Huse et al., 1989, “Generation of a Large Combinational Library of the Immunoglobulin Repertoire in Phage Lambda,” Science 246:1275-1281, L. Sastry et al., 1989 “Cloning of the Immunological Repertoire in Escherichia coli for Generation of Monoclonal Catalytic Antibodies: Construction of a Heavy Chain Variable Region-Specific cDNA Library,” Proc Natl. Acad. Sci USA 86:5728-5732; Kozbor et al., 1983 Immunol. Today 4, 72 re the human B-cell hybridoma technique; Cole et al. 1985 Monoclonal Antibodies in Cancer Therapy, Allen R. Bliss, Inc., pages 77-96 re the EBV-hybridoma technique to produce human monoclonal antibodies; and see also Michelle Alting-Mees et al., 1990 “Monoclonal Antibody Expression Libraries: A Rapid Alternative to Hybridomas,” Strategies in Molecular Biology 3:1-9). Hybridoma cells can be screened immunochemically for production of antibodies specifically reactive with an antigen, and monoclonal antibodies can be isolated.

The term “antibody” as used herein is intended to include antibody fragments which are specifically reactive with specific antigens on the surface of non-eosinophil cells. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

The invention also contemplates chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of differentiated cells or tumor cells. See, for example, Morrison et al., 1985; Proc. Natl. Acad. Sci. U.S.A. 81,6851; Takeda et al., 1985, Nature 314:452; Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B.

Antibodies may be selected for use in the antibody compositions of the invention based on their ability to deplete targeted non-eosinophils and recover non-targeted cells (i.e. eosinophils) in magnetic cell separations as more particularly described herein, and in U.S. Pat. No. 5,514,340, which is incorporated in its entirety herein by reference.

II. Process for Preparing Enriched Eosinophil Cell Preparations

The antibody compositions of the invention may be used to enrich and recover eosinophil cell preparations. The eosinophils may be enriched from many samples including peripheral blood and bronchio-alveolar lavage. When the sample is whole peripheral blood the invention may be used to target non-eosinophils for removal or preferably, the sample is first separated using density separation to remove the majority of the mononuclear cells. After centrifugation the pellet generally contains erythrocytes, granulocytes and mononuclear cells. After resuspension and lysis of the erythrocytes the sample generally contains 90% neutrophils, 5% eosinophils and 5% mononuclear cells. The sample is then treated as described below to enrich for eosinophils.

In accordance with a process of the invention, the sample is reacted with an antibody composition containing antibodies which are specific for selected antigens on the surface of the non-eosinophil cells to be removed from the sample and not on the eosinophils to be enriched in the sample, under suitable conditions, conjugates form between the antibodies contained in the antibody composition and the cells in the sample containing the antigens on their surface; and the conjugates are removed to provide a cell preparation enriched in eosinophils.

In one aspect the present invention provides a negative selection process for enriching and recovering eosinophils in a sample comprising (1) reacting the sample with an antibody composition containing antibodies capable of binding to the antigens (a) CD16; (b) CD2 and/or CD3 and/or both CD4 and CD8; (c) CD19 and/or CD20 and/or CD21 and/or CD22 and/or CD24 and/or IgG; and (d) CD14 and/or CD36, under conditions so that conjugates are formed between the antibodies and cells in the sample containing the antigens (a) CD16; (b) CD2 and/or CD3 and/or both CD4 and CD8; (c) CD19 and/or CD20 and/or CD21 and/or CD22 and/or CD24 and/or IgG; and (d) CD14 and/or CD36 on their surfaces; (2) removing the conjugates; and, (3) recovering a cell preparation which is enriched in eosinophils.

In the above negative selection processes of the invention for eosinophil cell enrichment, conditions which permit the formation of conjugates may be selected having regard to factors such as the nature and amounts of the antibodies in the antibody composition, and the estimated concentration of targeted cells in the sample.

The antibodies in the antibody compositions may be labelled with a marker or they may be conjugated to a matrix. Examples of markers are biotin, which can be removed by avidin bound to a support, and fluorochromes, e.g. fluorescein, which provide for separation using fluorescence activated sorters. Examples of matrices are magnetic beads, which allow for direct magnetic separation (Kernshead 1992), panning surfaces e.g. plates, (Lebkowski, J. S, et al., (1994), J. of Cellular Biochemistry supple. 18b:58), dense particles for density centrifugation (Van Vlasselaer, P., Density Adjusted Cell Sorting (DACS), A Novel Method to Remove Tumor Cells From Peripheral Blood and Bone Marrow StemCell Transplants. (1995) 3rd International Symposium on Recent Advances in Hematopoietic Stem Cell Transplantation-Clinical Progress, New Technologies and Gene Therapy, San Diego, CA), dense particles alone (Zwerner et al., Immunol. Meth. 1996 198(2):199-202) adsorption columns (Berenson et al. 1986, Journal of Immunological Methods 91:11-19.), and adsorption membranes. The antibodies may also be joined to a cytotoxic agent such as complement or a cytotoxin, to lyse or kill the targeted differentiated or tumors cells.

The antibodies in the antibody compositions may be directly or indirectly coupled to a matrix. For example, the antibodies in the compositions of the invention may be chemically bound to the surface of magnetic particles for example, using cyanogen bromide. When the magnetic particles are reacted with a sample, conjugates will form between the magnetic particles with bound antibodies specific for antigens on the surfaces of the non-eosinophil cells, and the non-eosinphil cells having the antigens on their surfaces.

Alternatively, the antibodies may be indirectly conjugated to a matrix using antibodies. For example, a matrix may be coated with a second antibody having specificity for the antibodies in the antibody composition. By way of example, if the antibodies in the antibody composition are mouse IgG antibodies, the second antibody may be rabbit anti-mouse IgG.

The antibodies in the antibody compositions may also be incorporated in antibody reagents which indirectly conjugate to a matrix. Examples of antibody reagents are bispecific antibodies, tetrameric antibody complexes, and biotinylated antibodies.

Bispecific antibodies contain a variable region of an antibody in an antibody composition of the invention, and a variable region specific for at least one antigen on the surface of a matrix. The bispecific antibodies may be prepared by forming hybrid hybridomas. The hybrid hybridomas may be prepared using the procedures known in the art such as those disclosed in Staerz & Bevan, (1986, PNAS (USA) 83: 1453) and Staerz & Bevan, (1986, Immunology Today, 7:241). Bispecific antibodies may also be constructed by chemical means using procedures such as those described by Staerz et al., (1985, Nature, 314:628) and Perez et al., (1985 Nature 316:354), or by expression of recombinant immunoglobulin gene constructs.

A tetrameric immunological complex may be prepared by mixing a first monoclonal antibody which is capable of binding to at least one antigen on the surface of a matrix, and a second monoclonal antibody from the antibody composition of the invention. The first and second monoclonal antibody are from a first animal species. The first and second antibody are reacted with an about equimolar amount of monoclonal antibodies of a second animal species which are directed against the Fc-fragments of the antibodies of the first animal species. The first and second antibody may also be reacted with an about equimolar amount of the F(ab′)₂ fragments of monoclonal antibodies of a second animal species which are directed against the Fc-fragments of the antibodies of the first animal species. (See U.S. Pat. No. 4,868,109 to Lansdorp, which is incorporated herein by reference for a description of tetrameric antibody complexes and methods for preparing same).

The antibodies of the invention may be biotinylated and indirectly conjugated to a matrix which is labelled with (strept) avidin. For example, biotinylated antibodies contained in the antibody composition of the invention may be used in combination with magnetic iron-dextran particles that are covalently labelled with (strept) avidin (Miltenyi, S. et al., Cytometry 11:231, 1990). Many alternative indirect ways to specifically cross-link the antibodies in the antibody composition and matrices would also be apparent to those skilled in the art.

In an embodiment of the invention, the cell conjugates are removed by magnetic separation using magnetic particles. Suitable magnetic particles include particles in ferrofluids and other colloidal magnetic solutions. “Ferrofluid” refers to a colloidal solution containing particles consisting of a magnetic core, such as magnetite (Fe₃O₄) coated or embedded in material that prevents the crystals from interacting. Examples of such materials include proteins, such as ferritin, polysaccharides, such as dextrans, or synthetic polymers such as sulfonated polystyrene cross-linked with divinylbenzene. The core portion is generally too small to hold a permanent magnetic field. The ferrofluids become magnetized when placed in a magnetic field. Examples of ferrofluids and methods for preparing them are described by Kemshead J. T. (1992) in J. Hematotherapy, 1:35-44, at pages 36 to 39, and Ziolo et al. Science (1994) 257:219 which are incorporated herein by reference. Colloidal particles of dextran-iron complex are preferably used in the process of the invention. (See Molday, R. S. and McKenzie, L. L. FEBS Lett. 170:232, 1984; Miltenyi et al., Cytometry 11:231, 1990; and Molday, R. S. and MacKenzie, D., J. Immunol. Methods 52:353, 1982; Thomas et al., J. Hematother. 2:297 (1993); and U.S. Pat. No. 4,452,733, which are each incorporated herein by reference).

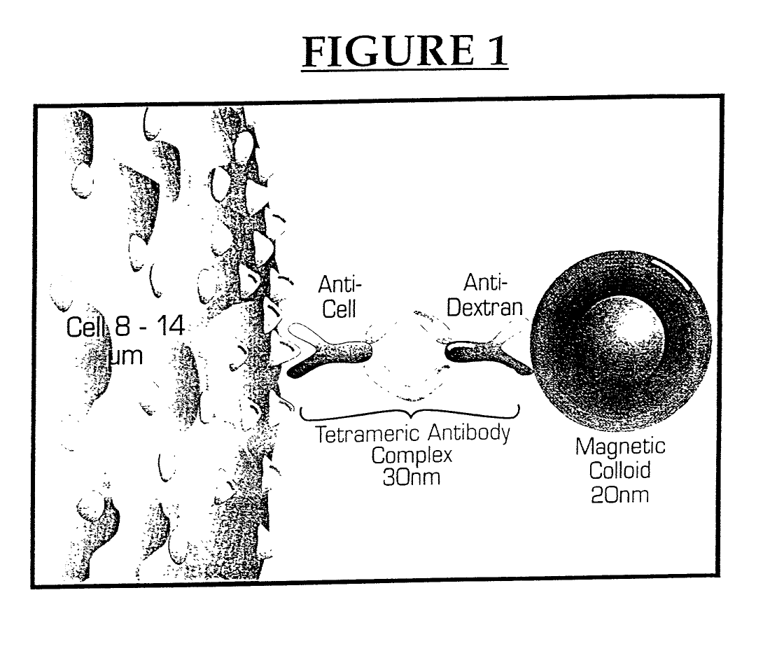

FIG. 1 is a schematic representation of magnetic cell labeling using tetrameric antibody complexes and colloidal dextran iron.

In accordance with the magnetic separation method, the sample containing the eosinophils to be recovered, is reacted with the above described antibody reagents, preferably tetrameric antibody complexes, so that the antibody reagents bind to the non-eosinophil cells present in the sample to form cell conjugates of the targeted non-eosinophil cells and the antibody reagents. The reaction conditions are selected to provide the desired level of binding of the targeted non-eosinophil cells and the antibody reagents. Preferably the sample is incubated with the antibody reagents for a period of 5 to 60 minutes at either 4° or ambient room temperature. The concentration of the antibody reagents is selected depending on the estimated concentration of the targeted differentiated cells in the sample. Generally, the concentration is between about 0.1 to 50 μg/ml of sample. The magnetic particles are then added and the mixture is incubated for a period of about 5 minutes to 30 minutes at the selected temperature. The sample is then ready to be separated over a magnetic filter device. Preferably, the magnetic separation procedure is carried out using the magnetic filter and methods described in U.S. Pat. No. 5,514,340 to Lansdorp and Thomas which is incorporated in its entirety herein by reference.

The sample containing the magnetically labelled cell conjugates is passed through the magnetic filter in the presence of a magnetic field. In a preferred embodiment of the invention, the magnet is a dipole magnet with a gap varying from 0.3 to 3.0 inches bore and having a magnetic field of 0.5-2 Tesla. The magnetically labelled cell conjugates are retained in the high gradient magnetic column and the materials which are not magnetically labelled flow through the column after washing with a buffer.

The preparation containing non-magnetically labelled cells may be analyzed using procedures such as flow cytometry.

The following non-limiting examples are illustrative of the present invention:

### EXAMPLES

### Example 1

Method for Evaluating Antibody Combinations

Whole peripheral blood is diluted with equal volume of PBS without Mg or Ca and layered over an equal volume of Ficoll Hypaque density solution (Pharmacia, Uppsala Sweeden). The layered sample is then centrifuged for 20 minutes at 1200× g, room temperature with the centrifuge brake off. Discard supernatant, plasma, and ficoll. Recover the cell pellet and resuspend in erythrocyte lysis buffer. The lysed pellet is then washed with 5-10 volume of PBS plus 2% fetal bovine serum (FBS) and labelled with tetrameric antibodies and colloidal dextran iron for magnetic cell depletions. Monoclonal antibodies recognizing specific cell surface antigens were mixed with a mouse IgG₁ anti-dextran antibody (Thomas, T. E, et al. (1992), J. Immunol Methods 154:245;252) and a rat IgG₁ monoclonal antibody which recognizes the Fc portion of the mouse IgG₁ molecule (TFL-P9) (Lansdorp, P. M, and Thomas, T. E. (1990), Mol. Immunol. 27:659-666). Tetrameric antibody complexes (Lansdorp, P. M, and Thomas, T. E. (1990), Mol. Immunol. 27:659-666; U.S. Pat. No. 4,868,109 to Lansdorp) spontaneously form when mouse IgG₁ molecules (the lineage specific monoclonal antibody and anti-dextran) are mixed with P9. A proportion of these tetrameric antibody complexes are bifunctional, recognizing an antigen on the surface of the target cell on one side and dextran (part of the magnetic colloidal dextran iron) on the other. Tetrameric antibody complexes were made for all the antibodies in the lineage cocktail.

Cells were labelled for separation (1-5×10⁷ cells/ml) by incubating them with the desired combination of tetramers for 30 min on ice or 15 minutes at room temperature followed by incubation for 30 minutes on ice or 15 minutes at room temperature with colloidal dextran iron (final OD450=0.6) (Molday and MacKenzie 1982, 52(3): 353-367). The cells were then passed through a magnetic filter (U.S. Pat. No. 5,514,340; inventors Lansdorp and Thomas) at 1 cm/min. The magnetically labelled cells bind to the filter and the unlabeled cells pass through. FIG. 1 shows a schematic representation of magnetic cell labeling using tetrameric antibody complexes and colloidal dextran iron.

The flow through fraction is collected and analyzed for eosinophils.

### Example 2

Enrichment of Eosinophils from a Suspension of Cells which Pellet in a Ficoll Density Separation

This example demonstrates the enrichment eosinophils using the method described in Example 1. Cells were labeled with a cocktail of tetrameric antibody complexes recognizing CD16 alone or a combination of CD2, CD14, CD16, CD19, CD56, and glycophorin A. The results, shown in Table 1, demonstrate that the method of the invention gives greater enrichment and recovery of eosinophils than depletion with anti-CD16 alone.

While what is shown and described herein constitutes various preferred embodiments of the subject invention, it will be understood that various changes can be made to such embodiments without departing from the subject invention, the scope of which is defined in the appended claims.

TABLE 1
Enrichment of Eosinophils from a Suspension of Cells which
Pellet in a Ficoll Density Separation

| | | % | % Recovery of |
|---|---|---|---|
| Sample | Cocktail | Eosinophils | Eosinophils |
| 1 | Anti-CD2, CD14, CD16, CD19, | 91 | 24 |
| | CD56, glycophorin A | | |
| | Anti-CD16 | 65 | 22 |
| 2 | Anti-CD2, CD14, CD16, CD19, | 98 | 45 |
| | CD56, glycophorin A | | |
| | Anti-CD16 | 95 | 23 |
| 3 | Anti-CD2, CD14, CD16, CD19, | 99 | 28 |
| | CD56, glycophorin A | | |
| | Anti-CD16 | 85 | 18 |
| 4 | Anti-CD2, CD14, CD16, CD19, | 100 | 58 |
| | CD56, glycophorin A | | |
| | Anti-CD16 | 94 | 59 |
| 5 | Anti-CD2, CD14, CD16, CD19, | 87 | 20 |
| | CD56, glycophorin A | | |
| 6 | Anti-CD16 | 77 | 19 |
| 7 | Anti-CD16 | 25 | 21 |
| 8 | Anti-CD16 | 85 | 35 |
| 9 | Anti-CD16 | 44 | 70 |

We claim:

1. An antibody composition for enriching for eosinophils comprising antibodies specific for the antigens (a) CD16; (b) CD2 and/or CD3 and/or both CD4 and CD8; (c) CD19 and/or CD20 and/or CD21 and/or CD22 and/or CD24 and/or IgG; and (d) CD14 and/or CD36.

2. An antibody composition according to claim 1 comprising antibodies specific for the antigens (a) CD16; (b) CD2; (c) CD19 and (d) CD14.

3. An antibody composition according to claim 2 further comprising antibodies specific for the antigens CD56 or glycophorin A.

4. An antibody composition according to claim 1 wherein the antibodies are monoclonal antibodies.

5. An antibody composition according to claim 4 wherein the antibodies are labelled with a marker or they are directly or indirectly conjugated to a matrix.

6. An antibody composition according to claim 4 wherein the antibodies are labelled with biotin or a fluorochrome.

7. An antibody composition according to claim 5 wherein the matrix is magnetic beads, a panning surface, dense particles for density centrifugation, an adsorption column, or an adsorption membrane.

8. An antibody composition according to claim 4 wherein each of the monoclonal antibodies is incorporated in a tetrameric antibody complex which comprises a first monoclonal antibody of a first animal species from the antibody composition according to claim 1, and a second monoclonal antibody of the first animal species which is capable of binding to at least one antigen on the surface of a matrix, which have been conjugated to form a cyclic tetramer with two monoclonal antibodies of a second animal species directed against the Fc-fragments of the antibodies of the first animal species.

9. A negative selection process for enriching and recovering eosinophils cells in a sample comprising (1) reacting the sample with an antibody composition containing antibodies capable of binding to the antigens (a) CD16; (b) CD2 and/or CD3 and/or both CD4 and CD8; (c) CD19 and/or CD20 and/or CD21 and/or CD22 and/or CD24 and/or IgG; and (d) CD14 and/or CD36, under conditions so that conjugates are formed between the antibodies and cells in the sample containing the (a) CD16; (b) CD2 and/or CD3 and/or both CD4 and CD8; (c) CD19 and/or CD20 and/or CD21 and/or CD22 and/or CD24 and/or IgG; and (d) CD14 and/or CD36, on their surfaces; (2) removing the conjugates; and (3) recovering a cell preparation which is enriched in eosinophils.

10. A negative selection method according to claim 9 wherein the antibody composition comprises antibodies specific for the antigens (a) CD16; (b) CD2; (c) CD19 and (d) CD14.

11. A negative selection method according to claim 10 wherein the antibody composition further comprises antibodies specific for the antigens CD56 or glycophorin A.

12. A process according to claim 9 wherein the antibodies in the antibody composition are monoclonal antibodies.

13. A process according to claim 12, wherein the antibodies in the antibody composition are labelled with a marker or they are conjugated to a matrix.

14. A process according to claim 12, wherein the antibodies in the antibody composition are labelled with biotin or a fluorochrome.

15. A process according to claim 12, wherein the matrix is magnetic beads, a panning surface, dense particles for density centrifugation, an adsorption column, or an adsorption membrane.

16. A process according to claim 12, wherein each of the monoclonal antibodies in the antibody composition is incorporated in a tetrameric antibody complex which comprises a first monoclonal antibody of a first animal species from the antibody composition according to claim 4, and a second monoclonal antibody of the first animal species which is capable of binding to at least one antigen on the surface of a matrix, which have been conjugated to form a cyclic tetramer with two monoclonal antibodies of a second animal species directed against the Fc-fragments of the antibodies of the first animal species.

17. A kit useful in preparing a cell preparation enriched in eosinophils comprising antibodies specific for the antigens (a) CD16; (b) CD2 and/or CD3 and/or both CD4 and CD8; (c)CD19 and/or CD20 and/or CD21 and/or CD22 and/or CD24 and/or Ig; and (d) CD14 and/or CD36, and instructions for preparing a cell preparation enriched in eosinophils.

18. A kit according to claim 17 comprising antibodies specific for the antigens (a) CD16; (b) CD2; (c) CD19 and (d) CD14.
